(19) United States
(12) Patent Application Publication
(10) Pub. No.: US 2010/0152728 A1
(43) Pub. Date: Jun. 17, 2010

(54) METHOD AND APPARATUS FOR DETERMINING THE EFFICACY OF A LESION

(76) Inventors: Christopher J. Park, Oregonia, OH (US); Jason L. Glithero, Mason, OH (US); Salvatore Privitera, Mason, OH (US)

Correspondence Address: COOK ALEX LTD, SUITE 2850, 200 WEST ADAMS STREET, CHICAGO, IL, 60606, US

(21) Appl. No.: 12/635,170

(22) Filed: Dec. 10, 2009

Related U.S. Application Data
(60) Provisional application No. 61/121,586, filed on Dec. 11, 2008.

(51) Int. Cl. A61B 18/14; A61B 5/0402

(52) U.S. Cl. 606/41; 600/509

(57) ABSTRACT

A method and apparatus are provided for assessing the degree of electrical signal blockage, or transmurality, of a line of ablation in which at least a first electrode adapted to deliver an electrical impulse is located on the first or one side of a line of ablation; and at least a second and a third electrode adapted to detect an electrical impulse are located on the second side of the line of ablation generally opposite to the first electrode. Each electrode may also be made up of more than one electrode, such as an electrode pair, to better assure reliability and tissue contact. Once the electrodes are located in contact with the tissue in question, an electrical impulse is delivered to the target tissue by the first electrode and detected by the second and third electrodes. Depending upon whether the line of ablation allows passage of electrical pulses, the second and third electrodes will detect the electrical impulse sequentially, with the order and/or timing of detection depending upon whether the electrical signal is able to directly cross the line of ablation or has to travel around the line of ablation to reach the second and third electrodes.

### CROSS-REFERENCE TO RELATED APPLICATION

This application claims the benefit of the filing date of U.S. provisional patent application Ser. No. 61/121,586 filed Dec. 11, 2008, the entire contents of which is incorporated herein by reference.

### BACKGROUND

Methods and apparatus have been developed for treating atrial fibrillation by creating lines of ablation or scar tissue that are intended to pose an interruption in the path of errant electrical impulses in the heart tissue. Scar tissue may be created by, e.g., surgical cutting of the tissue, freezing of the tissue by cryogenic probe, heating the tissue via RF energy and other technologies. Methods and apparatus for creating transmural lines of scar tissue or ablation using RF energy are shown and described in, e.g., U.S. Pat. No. 6,517,536, U.S. Pat. No. 6,974,454, and U.S. Pat. No. 7,393,353, which are incorporated herein by reference. The effectiveness of the ablation line at interrupting transmission of electrical signals is often associated, at least in part, with the transmurality of the line of ablation or scar tissue. Complete transmurality (fully through the thickness of the tissue) provides the most effective barrier. Partial or insufficient transmurality can allow undesired passage of aberrant electrical pulses.

Various methods for determining the efficacy of the lines of ablation have been developed using pacing and sensing electrodes. For example, U.S. Pat. No. 6,905,498, also incorporated herein by reference, discloses an RF ablation clamp in which the jaws of the clamp have a pacing electrode positioned so as to be on one side of a line of ablation formed by the instrument and a sensing or EKG electrode positioned on a jaw on the opposite side of the line of ablation. Accordingly, if a pacing pulse or signal is applied to cardiac tissue on one side of the line of ablation, but not sufficiently detected by the EKG sensor on the other side of the line of ablation, the line of ablation may be deemed effective for blocking the errant electrical impulses associated with atrial fibrillation.

### SUMMARY

The present application discloses a method and apparatus for determining the efficacy of a lesion for blocking electrical signals with the use of a plurality of electrodes. More specifically, a method is provided for assessing the degree of electrical signal blockage, or transmurality, of a line of ablation in which at least a first electrode adapted to deliver an electrical impulse is located on the first or one side of a line of ablation. At least second and third electrodes adapted to detect an electrical impulse are located on the second side of the line of ablation generally opposite to the first electrode, with the second electrode being spaced from the line of ablation a first distance and the third electrode being spaced from the line of ablation a second distance greater than the first distance. Each electrode may also be made up of more than one electrode, such as an electrode pair, to better assure reliability and tissue contact.

Once the electrodes are located in contact with the tissue in question, an electrical impulse is delivered to the target tissue by the first electrode (also called the pacing or stimulus electrode) and detected by the second and third electrodes (also called the EKG or sensing electrodes). Depending upon whether the line of ablation allows passage of electrical pulses, the second and third electrodes will detect the electrical impulse sequentially, with the order and/or timing of detection depending upon whether the electrical signal is able to directly cross the line of ablation or has to travel around the line of ablation to reach the second and third electrodes. Specifically, if the second electrode that is located closer to the line of ablation detects the electrical impulse prior to it being detected by the third electrode, then the line of ablation is not sufficiently transmural to block errant electrical impulses, as the electrical impulse has traveled directly across the line of ablation from the first electrode to the second electrode and then to the third electrode. On the other hand, if the third electrode detects the electrical impulse prior to it being detected by the second electrode, then the line of ablation is effective for directly blocking errant electrical impulses, as the electrical impulse has had to travel along or around the heart until it bypasses the ablation line to reach the second and third electrodes, and not through the line of ablation.

In another aspect, a device is provided for performing the method. The illustrated device preferably comprises a deflectable, steerable end effector having at least first, second and third electrodes located thereon for contacting the tissue. The second and third electrode comprise a first group or array of electrodes, with the distance between the first electrode and the first group or array of electrodes (comprising the second and third electrodes) being sufficient to accommodate therebetween a line of ablation made in the tissue. The second and third electrodes are adapted for detecting an electrical impulse while the first electrode is adapted for delivering an electrical impulse. Preferably, an analyzer with associated display or user interface is provided for determining and providing an indication of the activation sequence and or timing of the second and third electrodes in response to an electrical impulse delivered by the first electrode.

In a further aspect, a fourth electrode is provided adjacent the first electrode such that the first and fourth electrodes comprise a second group or array of electrodes. The first group or array of electrodes is spaced from the second group or array of electrodes a distance sufficient to accommodate therebetween the line ablation in the tissue. In this embodiment, the electrodes in each group are selectively either electrodes adapted for detecting electrical impulse or electrodes adapted for delivering an electrical impulse. Once again, an analyzer with an associated display or user interface is provided that is adapted to determine and provide an indication of the activation sequence of the electrodes in the group that is adapted for detecting the electrical impulse delivered by at least one of the electrodes in the group that is adapted to deliver an electrical impulse. By means of this embodiment, the efficacy of a lesion may be tested bi-directionally (i.e., with either group of electrodes being used for delivering or detecting electrical impulses), without the need to relocate or reposition the end effector.

In a preferred embodiment, each electrode comprises a pair of closely-spaced electrodes. In a specific embodiment, each electrode has a width of approximately 1 mm, with the electrodes in each pair being spaced approximately 1.5 mm. The pair of electrodes in each group are spaced approximately 10 mm apart, while the first group of electrodes are spaced approximately 15 mm from the second group of electrodes.

### DESCRIPTION OF THE DRAWINGS

FIG. 1 is a plan view of and exemplary device in accordance with the present disclosure.

FIG. 2 is an enlarged view of the end effector of the device of FIG. 1, showing one embodiment of an electrode configuration in accordance with the present disclosure.

FIG. 3 is a schematic view of a system including a device such as that illustrated in FIGS. 1 and 2 and an associated analyzer.

FIG. 4 is a schematic diagram of a switching circuit that may be used with the disclosed device.

FIG. 5 is a schematic depiction of the use of a device in accordance with the present disclosure to determine the efficacy (i.e., the transmurality) of a lesion made in heart tissue.

### DETAILED DESCRIPTION

In accordance with the present disclosure, a method and a device are provided for determining the efficacy of a lesion for blocking electrical impulses. The method and device preferably utilizes at least three electrodes (and preferably at least three pairs of closely-spaced electrodes) to determine the efficacy of a lesion. This is done by locating one of the electrodes (or electrode pairs) on one side of the lesion and the other two electrodes (or electrode pairs) on the other side of the lesion and using the time it takes for an electrical impulse generated by the one electrode to be detected by the other two electrodes to provide an indication of the efficacy of the lesion for blocking electrical impulses. The sequence and/or timing of activation of the second and third electrodes indicates the direction the electrical impulse has had to travel—either directly across the line of ablation (if the lesion is not sufficiently transmural) or in generally the opposite direction around the heart (if the lesion is sufficiently transmural).

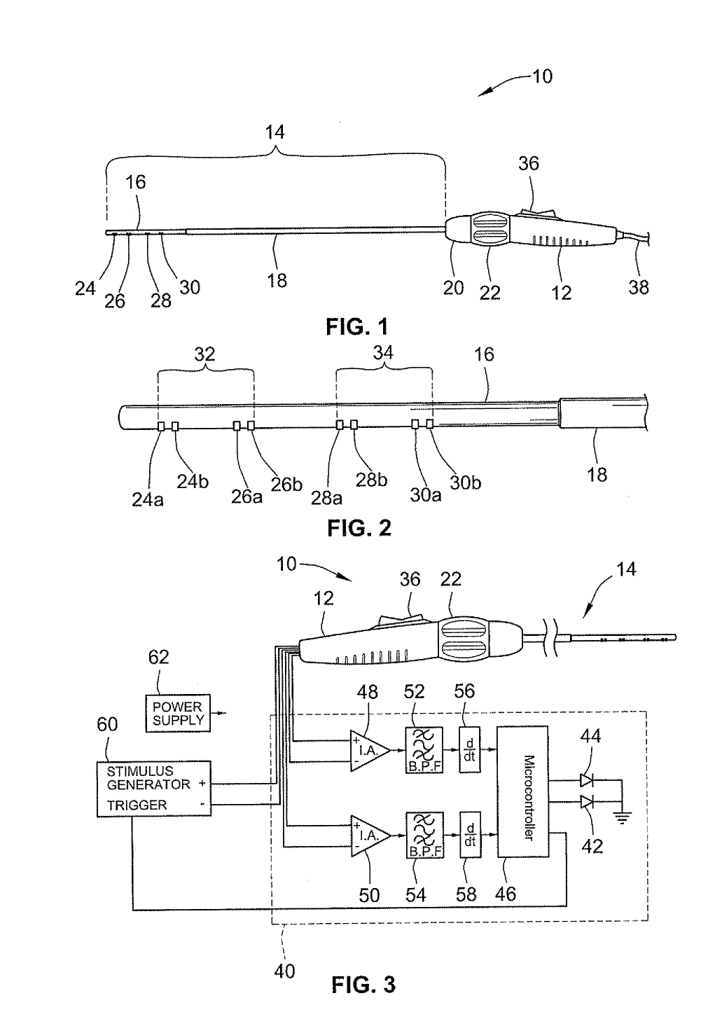

Turning to FIG. 1, there is seen a device, generally indicated 10, in accordance with the present disclosure. The device 10 includes a handle 12 to which is mounted an elongated shaft 14 having an end effector 16 at or near its distal end. In one example, the shaft 14 has an overall length of approximately 33 cm, with the proximal 25 cm preferably being rigid and the distal 7.5 cm comprising a deflectable section. The shaft may include a rigid portion 18 that comprises stainless steel or other material that is provided with an insulative finish or coating applied to the surface, or with a separate insulative overcoating or tube. The proximal end of the rigid portion of the shaft is secured to the handle 12 by a nose cone 20. The distal flexible or deflectable section 16 may be made of any suitable material, such as a polymer or other elastomer such as a thermoplastic elastomer, and, for example, a material such as Pebax brand block copolymer from Arkema Inc. of Philadelphia, Pa.

In one embodiment, the end effector 16 is sufficiently flexible so as to be capable of forming a two-inch diameter, 150° arc over its 7.5 cm of length, with the bending resistance preferably being substantially constant throughout the range of motion. The end effector 16 may be deflectable in any suitable manner and, as illustrated, is deflectable by means of a rotatable knob 22 on the handle 12 that may cooperate with one or more pull wires that extend through the shaft 14, as is generally known in the art. Preferably, a two pull wire system is used for steering, with a first pull wire to control the radius of curvature of the end effector 16 and a second pull wire to pivot the end effector 16 with respect to a longitudinal axis.

In one preferred embodiment, the end effector 16 is provided with four electrodes (or electrode pairs) 24, 26, 28, and 30 for contacting the tissue to be tested. However, the number of electrodes (or electrode pairs) may be varied. While four electrodes are shown in the embodiment of FIG. 1, the method and device of the present disclosure may be practiced with as few as three electrodes (or electrode pairs). More than four electrodes (or electrode pairs) may also be used. If more than four electrodes (or electrode pairs) are used, the device may be able to test more than one lesion for transmurality without having to reposition the end effector to relocate the electrodes. Preferably, the deflectable portion of the end effector has markings or indicators of a suitable type at the locations of the electrodes that provide the user with a direct visual indication of the position of the electrodes relative to the line of ablation.

With reference to FIG. 2, and as explained earlier, each electrode may be made up of a plurality of electrodes or electrical contact surfaces, such as a pair of electrodes or electrical surfaces, to better assure adequate tissue contact. Specifically, each electrode 24, 26, 28, 30 preferably comprises a pair of electrodes or electrical surfaces 24a, b, 26a, b, 28a, b, and 30a, b. Each electrode is preferably made of stainless steel with a polished finish and extends at least partially circumferentially about the end effector. As illustrated, the electrodes in each pair have a width of approximately 1 mm and are spaced approximately 1.5 mm from the other electrode in the pair. The electrode pairs comprise two arrays or groups 32, 34, with each electrode pair in each group being spaced from the pair in the same group such that the adjacent electrodes are approximately 10 mm apart. Further, the first group or array of electrode pairs 32 is spaced from the second group or array of electrode pairs 34 so as to accommodate a line of ablation in the tissue between. In the illustrated embodiment, the spacing between the adjacent electrodes 26b and 28a in the two groups is approximately 15 mm.

A control may be provided on the handle 12 for selecting which group of electrodes 32 or 34 will serve as the sending or pacing electrodes and as the receiving or EKG electrodes. The control may take the form of a rocker, toggle or slide switch 36 that may be thumb actuated. As illustrated, power is delivered to the pacing electrodes through a cord 38, which also houses the electrical conductors for transmitting the signals received by the EKG electrodes to an analyzer (seen in FIGS. 3 and 5). The analyzer determines the sequence and/or timing of activation of the EKG electrodes and provides an indication, preferably though a visual display, of the sequence or timing of activation, and may be either a separate unit into which the cord 38 is plugged or contained within the handle 12 and use a propriety software algorithm to determine directionality.

With reference to FIG. 3, the device 10 is shown in conjunction with a separate analyzer 40. The analyzer 40 preferably comprises a circuit (shown schematically) that interprets the signals generated and measured by the electrodes, and provides a signal to the user indicating whether a lesion has been created that satisfactorily blocks electrical impulses. For example, the analyzer 40 may be provided with a green light-emitting diode 42 that is illuminated if the lesion is “good” (i.e., the lesion blocks pacing signals) and a red light-emitting diode 44 that is illuminated if the lesion is “poor” (i.e., the lesion does not adequately block pacing signals). Preferably, if the contact of the electrodes to the epicardial surface of the heart is inadequate (i.e., not sufficiently conductive), neither diode will be illuminated. As can be appreciated, other types of signals, audible as well as visual, may be provided.

Returning to FIG. 3, a micro-controller 46 is provided that triggers a pacing or stimulus pulse from one electrode or electrode pair, and measures the arrival times of the reflex waves at the second and third electrodes or electrode pairs. As illustrated, the electrocardial signals are amplified by instrument amplifiers 48, 50. The resulting signals are then conditioned by band pass filters 52, 54 to remove interfering low frequencies, like power line hum, and interfering high frequencies like radio frequency noise. Thus, only signals containing valid EKG information are measured. The signals are then differentiated at 56, 58 to remove any DC offset and to produce sharp pulses for input to the micro-controller 46.

As shown, the stimulus/pacing generator 60 and power supply 62 are separate from the hand piece. Optionally, the stimulus/pacing generator and power supply may be incorporated into the hand piece, in which case the entire system becomes self-contained, and no connecting wires or cables or external power source are necessary. Under such circumstances, the power supply may be a non-replaceable battery that is connected to the system when, e.g., a tab is pulled, thus limiting the use of the device to a single surgical procedure.

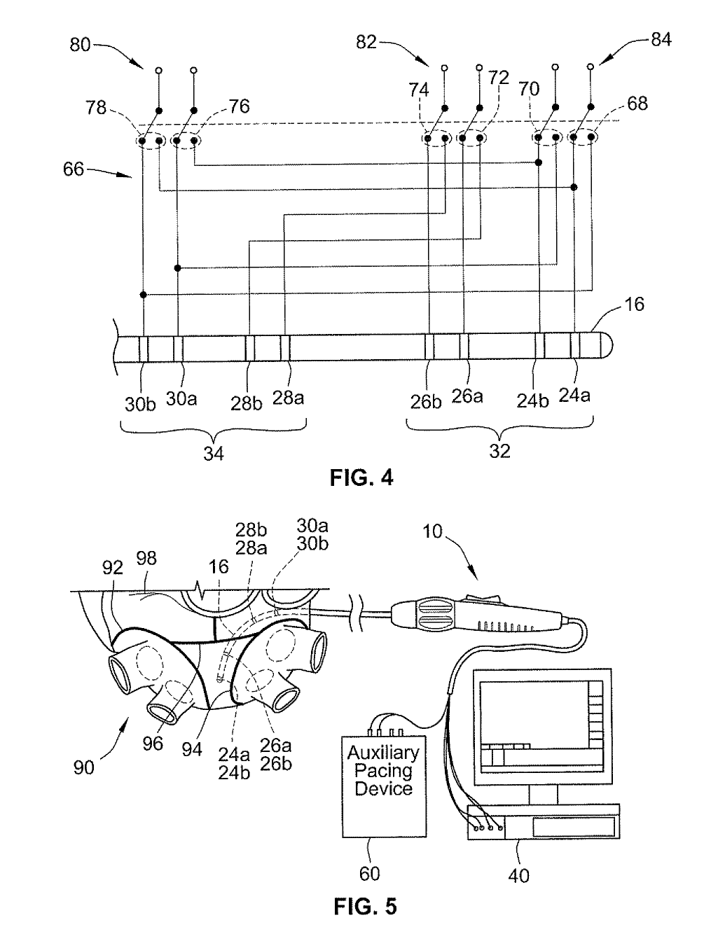

With reference to FIG. 4, a switching circuit 66 is shown that switches the four electrode pairs between six pairs of connecting wires 68, 70, 72, 74, 76, 78. The device 10 is capable of switching between the proximal electrodes and distal electrodes for the delivery of pacing signals, and, switching between the distal and proximal electrode pairs 32, 34 for the sensing of signals, by manipulating the switch 36 on the hand piece 12. Thus, the device may be operated without the necessity of disconnecting and reconnecting the wires from the electrodes to the analyzer.

Specifically, the circuit 66 includes a first switch 80 connected to the stimulus or pacing generator and selectively connecting either the proximal pair 30a, 30b or distal pair 24a, 24b of the electrodes thereto. A second switch 82 connects a first EKG channel to one of the two inner pairs of electrodes 26a, b or 28a, b, and a third switch 84 selectively connects a second EKG channel to one of the proximal and distal pairs of electrodes 24a, b or 30a, b. Operation of the three switches is simultaneous such that the electrodes connected to the pacing generator will be in one of the groups 32, 34 of electrodes and the two electrodes connected to the EKG channels will be in the other group of electrodes. By using the switch 36, the user will develop an intuitive feel for whether the pacing pulses are being applied to the distal or proximal electrodes, making a visual indication of such status unnecessary.

As an alternative, the switching circuit can be modified to provide for three pairs of sensing electrodes to be used in combination with either a single pair of pacing electrodes or an entirely separate and self-contained pacing device. The use of the third pair of sensing electrodes allows for the determination of the angle at which the pacing impulse is received. Specifically, the use of two sensing points indicates which one of two directions an electrical impulse is moving. The addition of the third sensing point, with the relative positions of the sensing points being fixed and the appropriate sensing equipment to measure the timing, allows the determination of the angle at which the electrical impulse is passing the sensing points.

One particular use of a device such as that described above to determine the efficacy of a lesion is shown in FIG. 5. Although illustrated with respect to heart tissue, other types of tissues could also be tested using the described apparatus and method. FIG. 5 depicts a human heart 90 in which a series of ablation lines have been made as part of the “Maze” procedure for treating atrial fibrillation. Lesions 92 and 94, respectively, separately encircle the left and right pulmonary veins. Lesion 96, called the “roof line lesion,” connects the lesions 92 and 94, while a lesion 98, called the “trigone lesion,” extends between the mitral valve annulus and the roof line lesion 96.

A device having an end effector 16 in accordance with the present disclosure is provided. The end effector 16 shown in FIG. 3 comprises four electrodes, each made up of a pair of electrodes, with electrode pairs 24a, b and 26a, b comprising the first array or group of electrodes and electrodes 28a, b and 30a, b comprising the second array or group of electrodes. The end effector 16 is brought into contact with the surface of the heart 90, with the first and second group of electrodes straddling the trigone lesion 98. To test the lesion in a first direction, the electrode pair 24a, b in the first group is switched so as to be connected to a pacing pulse generator 60, while the electrode pairs 28a, b and 30a, b serve as the EKG electrodes and are connected to the analyzer 40.

A pacing pulse is delivered to the heart 90 through the electrodes 24a, b. If the trigone lesion 98 is sufficiently effective to block electrical signals (i.e., transmural), the pacing pulse cannot cross and, in order to be detected by the EKG electrodes, must travel around the back of the heart, where it would first be detected by electrode pair 30a, b and later by electrode pair 28a, b. The analyzer 40 determines the sequence of activation of the electrode pairs, and provides an indication thereof. If the sequence of activation is first electrode pair 30a, b and then the electrode pair 28a, b, the lesion 98 is deemed transmural. If the sequence of activation is first electrode pair 28a, b and then electrode pair 30a, b, the lesion is deemed to be not transmural or not sufficiently formed to block electrical signals.

The lesion 98 may then be tested from the opposite direction without moving end effector in order to confirm the determination of transmurality. This may be accomplished by switching the device so that electrode pair 30a, b serves as the sending or pacing electrode, and the electrode pairs 24a, b and 26a, b serve as the receiving or EKG electrodes. Thus, the device may be used bi-directionally to test the transmurality of a lesion.

After a first lesion is tested, the end effector may be manipulated so that the first and second groups 32, 34 of electrodes straddle a different lesion, and the testing of that lesion is conducted as set forth above.

Thus, a method and apparatus for determining the efficacy of a lesion for blocking electrical signals. While the method and apparatus have been described in the context of certain preferred embodiments, there is no intent to limit this application to the same. Instead, the method and apparatus are to be defined by the following claims.

1. A device for measuring electrical impulses in tissue comprising:
an end effector;
a first, a second and a third electrode located on the end effector for contacting the tissue, the second and third electrode being spaced from the first electrode sufficiently to accommodate therebetween a line of ablation in the tissue, the second and third electrodes being adapted for detecting an electrical impulse and the first electrode being adapted for delivering an electrical impulse.

2. The device of claim 1 further comprising:
a fourth electrode adjacent the first electrode and located on the end effector such that the first and fourth electrodes are spaced from the second and third electrodes a distance sufficient to accommodate therebetween a line of ablation in the tissue, with the first and fourth electrodes on one side of the ablation and the second and third electrodes are on the other side of the ablation, the electrodes being selectively either electrodes adapted for detecting an electrical impulse or electrodes adapted for delivering an electrical impulse.

3. The device of claim 1 wherein the end effector is rotatable about a longitudinal axis therethrough and is deflectable in a plane including the longitudinal axis to facilitate bringing the electrodes into contact with the tissue.

4. The device of claim 3 for further comprising a handle and a rigid shaft, the shaft having a proximal end mounted to the handle and a distal end section carrying the end effector.

5. The device of claim 4 wherein the handle further comprises control for selecting the electrodes to deliver an electrical impulse, and the electrodes to detect an electrical impulse.

6. The device of claim 1 wherein each electrode has a width of approximately 1 mm, the electrodes in each group are spaced approximately 10 mm apart, and the groups of electrodes are spaced approximately 15 mm apart.

7. The device of claim 1 wherein the end effector has a circumference and the electrodes extend partially about the circumference of the end effector.

8. The device of claim 7 wherein the electrodes have a circumferential extent of at least about 130 degrees.

9. A system for measuring electrical impulses in tissue comprising:
a deflectable, steerable end effector;
a first, a second and a third pair of closely-spaced electrodes located on the end effector for contacting the tissue, the second and third pairs of electrodes being spaced relatively closer to each other than to the first pair of electrodes so as to comprise a first group of electrodes such that the distance between the first group of electrodes and the first pair of electrodes is sufficient to accommodate therebetween a line of ablation made in the tissue, the first group of electrodes being adapted for detecting an electrical impulse and the first pair of electrodes being adapted for delivering an electrical impulse;
an electrical pulse generator conductively connected to the first pair of electrodes; and
an analyzer for determining and providing an indication of the activation sequence of the second and third pairs of electrodes in response to an electrical impulse delivered by the first pair of electrodes.

10. The system of claim 9 further comprising:
a fourth pair of closely spaced electrodes adjacent the first pair of electrodes and located on the end effector such that the first and fourth pairs of electrodes comprise a second group of electrodes the first group of electrodes being spaced from the second group of electrodes a distance sufficient to accommodate therebetween a line of ablation in the tissue, the electrodes in each group being selectively either electrodes adapted for detecting an electrical impulse or electrodes adapted for delivering an electrical impulse; and
the analyzer is adapted to determine and provide an indication of the activation sequence of the pairs of electrodes in the group that is adapted for detecting an electrical impulse delivered by at least one of the electrode pairs in the group that is adapted to deliver an electrical impulse.

11. The system of claim 9 wherein the end effector is rotatable about a longitudinal axis therethrough and is deflectable in a plane including the longitudinal axis to facilitate bringing the electrodes into contact with the tissue.

12. The system of claim 11 for further comprising a handle and a rigid shaft, the shaft having a proximal end mounted to the handle and a distal end terminating in the end effector.

13. The system of claim 12 wherein the handle further comprises control for selecting one of the first group of electrodes and the second group of electrodes to be adapted to deliver an electrical impulse, and the other of the first group of electrodes and the second group of electrodes being adapted to detect an electrical impulse.

14. The system of claim 9 wherein each electrode has a width of approximately 1 mm, the electrodes in each pair are spaced approximately 1.5 mm apart, the pair of electrodes in each group are spaced approximately 10 mm apart, and the groups of electrodes are spaced approximately 15 mm apart.

15. The system of claim 9 wherein the end effector has a circumference and the electrodes extend partially about the circumference of the end effector.

16. The system of claim 15 wherein the electrodes have a circumferential extent of at least about 130 degrees.

17. A method for assessing the transmurality of a line of ablation in a target tissue comprising:
delivering an electrical impulse on a first side of the line of ablation in the tissue;
detecting an electrical impulse at first and second spaced-apart locations in the tissue on a second side of the line of ablation, the first location being spaced from the line of ablation a first distance and the second location being spaced from the line of ablation a second distance greater than the first distance;
comparing the sequence or time of detecting at the first and second locations in response to the electrical impulse to determine a characteristic of the line of ablation.

18. The method of claim 17 wherein the characteristic includes transmurality of the line of ablation.

19. The method of claim 17 further comprising detecting the electric impulse at a third location on the second side of the line of ablation spaced apart from the first and second spaced-apart locations and determining the angle at which the electrical impulses are received at the first, second and third spaced-apart locations.

20. The method of claim 17 wherein the electrical impulse is delivered and detected by first, second and third electrodes.

21. The method of claim 20 wherein each of the first, second and third electrodes are mounted on a single end effector.

22. The method of claim 20 wherein the electrode for delivering the electrical impulse is mounted on a first end effector and the electrodes for detecting the electrical impulse are mounted on a second end effector.

23. The method of claim 17 wherein a device is provided having first, second, third and fourth spaced-apart electrodes and the device includes a switch for selectively connecting one of the electrodes to a pulse generator and selectively adapting at least two of the electrodes to detect electrical impulses.
